SEQUENCE LISTING (18 sequences)

SEQ ID NO: 1 - DNA, 45 bp, Artificial Sequence
  Description of Artificial Sequence Synthetic primer
tcaaaacgatcctggaaaataatggacattcattctgagctgtgc

SEQ ID NO: 2 - DNA, 47 bp, Artificial Sequence
  Description of Artificial Sequence Synthetic primer
ggaaaaactaaatgagaatgtcaaggagaaaaaacattcttgacttc

SEQ ID NO: 3 - DNA, 18 bp, Artificial Sequence
  Description of Artificial Sequence Synthetic primer
ttcaggctcaccttgtga

SEQ ID NO: 4 - DNA, 18 bp, Artificial Sequence
  Description of Artificial Sequence Synthetic primer
ctgtggcaataaagcacc

SEQ ID NO: 5 - DNA, 20 bp, Artificial Sequence
  Description of Artificial Sequence Synthetic primer
agcagatgacattgcatgga

SEQ ID NO: 6 - DNA, 45 bp, Artificial Sequence; identical to SEQ ID NO: 1
  Description of Artificial Sequence Synthetic primer

SEQ ID NO: 7 - DNA, 47 bp, Artificial Sequence; identical to SEQ ID NO: 2
  Description of Artificial Sequence Synthetic primer

SEQ ID NO: 8 - DNA, 18 bp, Artificial Sequence
  Description of Artificial Sequence Synthetic primer
ttcaggctcaccttgtga

SEQ ID NO: 9 - DNA, 18 bp, Artificial Sequence
  Description of Artificial Sequence Synthetic primer
ctgtggcaataaagcacc

SEQ ID NO: 10 - DNA, 20 bp, Artificial Sequence
  Description of Artificial Sequence Synthetic primer
agcagatgacattgcatgga

SEQ ID NO: 11 - DNA, 45 bp, Artificial Sequence
  Description of Artificial Sequence Synthetic primer
atttggaacatactgctctcttctgctgccatcttccttttgaca

SEQ ID NO: 12 - DNA, 45 bp, Artificial Sequence
  Description of Artificial Sequence Synthetic primer
aactcagacctccttgaaaagaacacaaaatcctcgataactcgg

SEQ ID NO: 13 - DNA, 18 bp, Artificial Sequence
  Description of Artificial Sequence Synthetic primer
atctgggaaggatcagcc

SEQ ID NO: 14 - DNA, 22 bp, Artificial Sequence
  Description of Artificial Sequence Synthetic primer
tgtctgaagatagctattcaca

SEQ ID NO: 15 - DNA, 25 bp, Artificial Sequence
  Description of Artificial Sequence Synthetic primer
ctatttttgatcacattgtaagaag

SEQ ID NO: 16 - DNA, 18 bp, Artificial Sequence
  Description of Artificial Sequence Synthetic primer
gctctctcctgatttggt

SEQ ID NO: 17 - DNA, 23 bp, Artificial Sequence
  Description of Artificial Sequence Synthetic primer
atacttatttacgcttgaacctc

SEQ ID NO: 18 - DNA, 26 bp, Artificial Sequence
  Description of Artificial Sequence Synthetic primer
gttccttattctaaatagtaagggat

(19) United States
(12) Patent Application Publication
(10) Pub. No.: US 2010/0099094 A1
(43) Pub. Date: Apr. 22, 2010

(54) METHOD OF DETECTING NUCLEIC ACID AMPLIFICATION REACTION

(76) Inventors: Jun OKADA, Tokyo (JP); Nobuhiro Gemma, Yokohama-shi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/498,721

(22) Filed: Jul. 7, 2009

(30) Foreign Application Priority Data
Oct. 21, 2008 (JP) 2008-271228

(51) Int. Cl. C12Q 1/68

(52) U.S. Cl. 435/6

(57) ABSTRACT

The present invention provides a method of detecting a nucleic acid amplification reaction, including the steps of adding a sample nucleic acid to a nucleic acid amplification buffer containing a reducing agent molecule, a redox molecule and a magnesium ion, to conduct an amplification reaction, measuring a reduction current produced by a reduction reaction of the reducing agent molecule with the redox molecule, under the conditions that when the amplification reaction of the sample nucleic acid has proceeded in the buffer, pyrophosphoric acid formed with the progress of amplification of the sample nucleic acid forms magnesium pyrophosphate with the magnesium ion, thereby decreasing a magnesium ion concentration of the buffer, and determining, from the magnitude of the reduction current measured above, whether the sample nucleic acid has been amplified or not.

### CROSS-REFERENCE TO RELATED APPLICATIONS

This application is based upon and claims the benefit of priority from prior Japanese Patent Application No. 2008-271228, filed Oct. 21, 2008, the entire contents of which are incorporated herein by reference.

### BACKGROUND OF THE INVENTION

1. Field of the Invention

The present invention relates to a method of detecting a nucleic acid amplification reaction and in particular to a method of detecting, by an electrochemical technique, the presence or absence of nucleic acid amplification in the amplification reaction of a nucleic acid by an enzymatic reaction.

2. Description of the Related Art

With the development of molecular biology in recent years, many disease genes have been identified, and the identification of diseases by genetic diagnosis has become possible. Tailor-made medicine which, on the basis of results on genetic diagnosis, provides optimum treatment to each patient is being realized. Techniques of detecting viral genes have also been developed for diagnosis of infectious diseases. Necessity for genetic tests is increasing in food inspection, personal authentication, etc., besides the medical field.

To conduct genetic diagnosis, the concentration of nucleic acid in a sample is usually too low, thus making it necessary to conduct a step of nucleic acid amplification reaction with a sample nucleic acid as a template by using primer nucleic acids for causing a nucleic acid amplification reaction of a sequence to be detected. Generally, this step involves PCR, LAMP, ICAN or SMAP. However, when the sample nucleic acid does not have a sequence to be detected, no nucleic acid amplification reaction occurs, and therefore, subsequent genetic diagnosis becomes unnecessary. Whether the amplification reaction has occurred or not can be determined at present by electrophoresis following the nucleic acid amplification reaction. In LAMP, whether the amplification reaction has occurred or not can be determined by detecting the turbidity of precipitates of magnesium pyrophosphate formed as a byproduct in the amplification reaction. However, these techniques need a complicated process and a special apparatus, and thus simple techniques have been desired. Meanwhile, some techniques of determining, by an electrochemical method, whether the amplification reaction has occurred or not have been reported in recent years. The electrochemical method reported until now includes, for example, a method which includes adding a metal ion to an amplification reaction solution to form a complex compound and then measuring the electrochemical property of the complex compound (JP-A 2007-37483 (KOKAI)), a method of measuring a change in electrochemical property produced by oxidation of guanine bases constituting a sample nucleic acid (JP-A 2008-157733 (KOKAI)), and a method of electrochemically measuring the concentration of pyrophosphoric acid formed in a nucleic acid amplification process (JP-A 2007-295811 (KOKAI)).

### BRIEF SUMMARY OF THE INVENTION

The methods described in the patent documents mentioned above are those methods which include electrochemically oxidizing a compound contained in an amplification product and measuring its oxidation current. However, a nucleic acid is poor in oxidation resistance, so the nucleic acid in a sample solution may be decomposed by oxidation to make accurate measurement difficult.

In the method described in JP-A 2007-37483 supra, there is necessity for addition of a new reagent containing a metal ion, thus making a detection process complicated, and such metal ion may reduce a polymerase activity, to adversely affect the amplification reaction. In the method described in JP-A 2008-157733 supra, there is a problem that because a current to be detected varies significantly depending on the guanine content of a sample nucleic acid, the accuracy of detection is destabilized depending on a sequence of an amplification product, and because there is necessity for application of high voltage in the direction of oxidation, a highly reliable gold electrode with stable performance cannot be used. In the method described in JP-A 2007-295811 supra, a special pyrophosphate sensor electrode coated with a pyrophosphate detecting substance is necessary. For improving sensitivity, this sensor electrode is required to be molded in the form of a net or disk in order to increase the area of the electrode as a whole, thus being subject to a problem of a complicated structure of a device to increase the detection cost. In addition, troublesome coating of the electrode with a pyrophosphate detecting substance is necessary.

An object of the present invention is to provide an electrochemical detection method capable of solving these problems, that is, a constitutive and accurate detection method which requires neither addition of a reagent possibly influencing the amplification reaction nor a special sensor electrode having a complicated structure and which is not influenced by a sequence of a sample nucleic acid.

As a result of extensive study, the inventors focused attention on a new chemical species and found that the presence or absence of the amplification of a sample nucleic acid could be determined by measuring the reduction current of this chemical species.

That is, the present invention provides a method of detecting a nucleic acid amplification reaction, comprising the steps of: adding a sample nucleic acid to a nucleic acid amplification buffer containing a reducing agent molecule, a redox molecule and a magnesium ion, to conduct an amplification reaction; measuring a reduction current produced by a reduction reaction of the reducing agent molecule with the redox molecule, under the conditions that when the amplification reaction of the sample nucleic acid has proceeded in the buffer, pyrophosphoric acid formed with the progress of amplification of the sample nucleic acid forms magnesium pyrophosphate with the magnesium ion, thereby decreasing a magnesium ion concentration of the buffer; and determining, from the magnitude of the reduction current measured above, whether the sample nucleic acid has been amplified or not.

In the method of detecting a nucleic acid amplification reaction according to the present invention, a reduction current produced by the reduction reaction of a reducing agent molecule with a redox molecule, both of which are present in an amplification buffer, is measured and the voltage applied to a sample is relatively low, so that there does not occur the decomposition, with an oxidation current, of a nucleic acid poor in oxidation resistance, and accurate measurement can be realized. The method does not need addition, for detection of a reaction, of a reagent adversely affecting an amplification reaction itself and can thus realize simple measurement not adversely affecting the amplification reaction itself. Further, the method has detection accuracy not depending on the sequence of an amplification product. Furthermore, the method does not need application of a high voltage in the direction of oxidation, is thus not subject to limitation of the type of electrode used and can employ the most reliable gold electrode for example. The method deals with a reaction in solution and so needs neither expansion of the area of an electrode nor a special sensor electrode in the form of a net or disk, and can thus be carried out with an apparatus of simple structure to reduce the detection cost.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a graph showing a measurement result of a reduction current produced by the reduction reaction of a reducing agent molecule with a redox molecule;

FIG. 2 is a graph showing a measurement result of a reduction current produced by the reduction reaction of a reducing agent molecule with a redox molecule in the presence of magnesium ion;

FIG. 3 is a graph showing a measurement result of a reduction current produced by the reduction reaction in the first embodiment;

FIG. 4 is a table showing a series of chemical reaction formulae in the reduction reaction in the first embodiment;

FIG. 5 is a schematic diagram of a first measurement apparatus 20 used in the first embodiment;

FIG. 6 is a schematic diagram of a second measurement apparatus 50 used in the first embodiment;

FIG. 7 is a schematic diagram of a third measurement apparatus 100 used in the first embodiment;

FIG. 8 is a graph showing peak magnitudes of reduction currents obtained before and after amplification in Example 1;

FIG. 9 is a graph showing peak magnitudes of reduction currents obtained in amplification reaction solutions 1 to 4 in Example 2; and

FIG. 10 is a graph showing peak magnitudes of reduction currents obtained in amplification reaction solutions 5 and 6 in Example 3.

### DETAILED DESCRIPTION OF THE INVENTION

### First Embodiment

### (1) Preparation of Nucleic Acid Amplification Buffer

First, a nucleic acid amplification buffer is prepared. This buffer is an amplification buffer which contains not only a polymerase, a primer and an NTP substrate, but also a reducing agent molecule, a redox substance, and a magnesium ion. The former 3 substances are substances for amplifying a nucleic acid, and the latter 3 substances are substances for stabilizing a nucleic acid during amplification reaction. In addition, arbitrary components are contained appropriately in the buffer.

The amplification reaction in the first embodiment is not limited as long as it is an amplification reaction using a polymerase and NTP substrate, and this amplification reaction is selected for example from the group consisting of PCR, LAMP, ICAN and SMAP. Accordingly, the buffer contains a polymerase adapted to the amplification reaction to be carried out. For example, when the amplification reaction is conducted by PCR, a thermostable polymerase is used, and when the amplification reaction is conducted by LAMP, a strand displacement-type polymerase is used. The primer is designed appropriately depending on the amplification reaction to be carried out and the nucleic acid to be amplified.

The reducing agent molecule used in the first embodiment, although being not particularly limited, is selected for example from the group consisting of dithiothreitol, β-mercaptoethanol, SO₂ and H₂S, among which dithiothreitol and β-mercaptoethanol are preferable. These reducing agent molecules function in maintaining the activity of the polymerase in the amplification reaction and is a component contained usually in the nucleic acid amplification buffer. The concentration of the reducing agent molecule varies depending on the type of the molecule to be added; for example, when the reducing agent molecule is dithiothreitol, its concentration is preferably 1 μM to 100 mM, more preferably 5 μM to 10 mM.

The redox molecule used in the first embodiment should be a molecule having a property of being reduced only in the presence of the reducing agent molecule. This is to secure reaction specificity. The redox molecule used in the first embodiment should be a molecule having an unshared electron pair. This is to enable the redox molecule to give an electron to, or receive an electron from, the reducing agent molecule, thereby advancing the reduction reaction. Specifically, the redox molecule used in the first embodiment, although being not particularly limited, is selected for example from the group consisting of an ammonium ion, a molecule having a quinone group, an amido group, a carboxyl group or a hydroxyl group, a metal complex, and a molecule having an unshared electron pair, among which an ammonium ion is preferable. When an ammonium ion is used as the redox molecule, the ammonium ion is added appropriately in the form of a salt such as ammonium sulfate, ammonium nitrate, ammonium phosphate, or ammonium halide. These redox substances function in unwinding a double strand during amplification reaction to stabilize it, and is a component contained usually in the nucleic acid amplification buffer. The concentration of the redox molecule varies depending on the type of the molecule added; for example, when the redox molecule is an ammonium ion, its concentration is preferably 10 pM to 1M, more preferably 100 pM to 500 mM.

The magnesium ion used in the first embodiment, although being not particularly limited, is selected appropriately in the form of a salt such as magnesium sulfate, magnesium nitrate, magnesium phosphate or magnesium halide. The magnesium ion functions both in maintaining the activity of an enzyme during amplification reaction and in increasing the stability of a double strand during amplification reaction, and is a component contained usually in the nucleic acid amplification buffer. The concentration of the magnesium ion is preferably 10 pM to 1M, more preferably 100 pM to 500 mM.

### (2) Amplification of Sample Nucleic Acid

Then, a sample nucleic acid is added to the prepared amplification buffer and subjected to amplification reaction. Preferably, the sample nucleic acid has previously been prepared in a form adapted to the amplification reaction.

The sample nucleic acid to be examined in the first embodiment is not especially limited, and may be a nucleic acid extracted from a sample such as blood, serum, leukocyte, urine, feces, semen, saliva, tissue, cultivated cell, phlegm, food, soil, drainage, waste water, air, or the like.

The phrase “amplification reaction is conducted” means that after a sample nucleic acid is added, the amplification buffer is heated to a predetermined temperature. Heating control conditions vary depending on the selected amplification reaction, and heating control adapted to each amplification reaction is conducted. For example, when the amplification reaction is conducted by PCR, the temperature is changed alternately between thermal denaturation temperature (about 98° C.) and chain elongation reaction temperature (about 65° C.), and when the amplification reaction is conducted by LAMP, the temperature is kept constant at about 63° C.

The amplification reaction in the first embodiment is conducted under the conditions where when the amplification reaction has proceeded, pyrophosphoric acid formed with the progress of amplification of the sample nucleic acid binds to a magnesium ion in the reaction solution to form magnesium pyrophosphate, thereby reducing the magnesium ion concentration of the solution. These conditions are realized in the amplification reaction with the polymerase and NTP substrate in the presence of the magnesium ion contained in the amplification buffer.

### (3) Measurement of Reduction Current

(3-1) Principle of Measurement of Reduction Current

Subsequently, a reduction current produced by the reduction reaction of the reducing agent molecule with the redox molecule is measured.

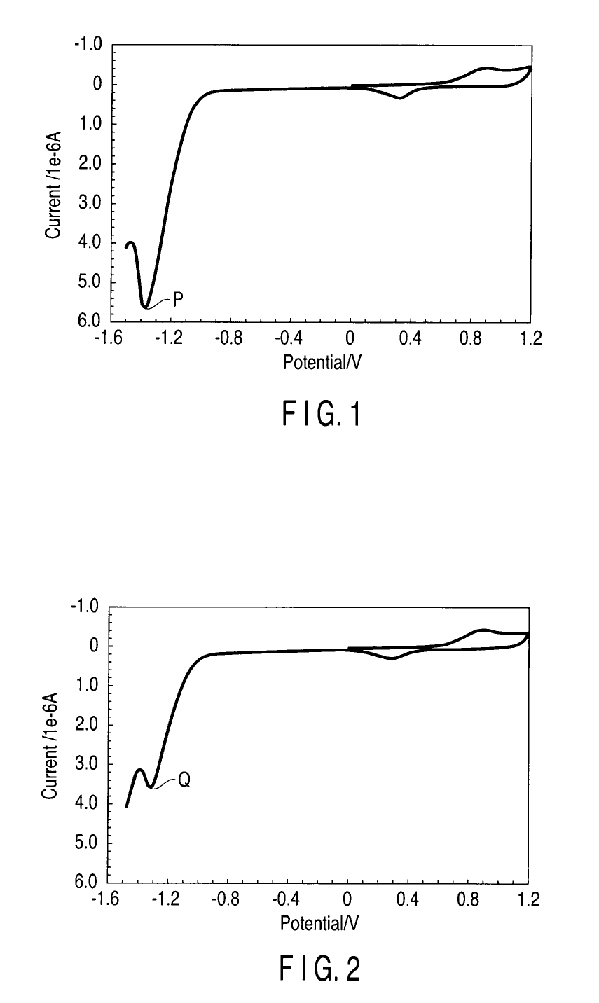

FIG. 1

FIG. 1 is a graph showing a measurement result of a reduction current produced by the reduction reaction of a reducing agent molecule with a redox molecule. Sweep potential is indicated on the abscissa and reduction current measurements on the ordinate. In measurement of reduction current, buffer A containing a reducing agent molecule and a redox molecule is added to an electrolyte solution (Tris-HCl) in a container provided with a gold electrode, and a reduction current produced between the two molecules is measured with the gold electrode by cyclic voltammetry. The reducing agent molecule used herein was dithiothreitol (DTT), and the redox molecule used was an ammonium ion (ammonium sulfate). The measurement result in FIG. 1 is a result obtained by sweeping the potential positively to 1.2V (through the upper branched current magnitude in FIG. 1) and then sweeping the potential negatively. The potential is swept in the direction of oxidation of an ammonium ion as the redox molecule, thereby sufficiently oxidizing the ammonium ion, and then the potential is swept in the direction of reduction of the ammonium ion, whereby the equilibrium state of the reduction reaction can be shifted maximally to the direction of reduction of the ammonium ion, and as a result a higher reduction current can be obtained. From FIG. 1, it can be seen that when the potential is swept to −1.3V or so, a current of 5.6 μA can be obtained as a peak current P.

FIG. 2

FIG. 2 is a graph showing a measurement result of a reduction current produced by the reduction reaction of a reducing agent molecule with a redox molecule in the presence of magnesium ion. In FIG. 2 similar to FIG. 1, sweep potential is indicated on the abscissa and reduction current measurements on the ordinate. In measurement of reduction current, buffer B containing not only a reducing agent molecule and a redox molecule but also magnesium sulfate is added to an electrolyte solution (Tris-HCl) in a container provided with a gold electrode, and a reduction current produced is measured with the gold electrode by cyclic voltammetry. The reducing agent molecule, the redox molecule and the potential sweeping method used herein are under the same conditions as in the graph in FIG. 1. Referring to FIG. 2, a peak current can be obtained when the potential is similarly swept to −1.3V or so, and its magnitude Q is 3.6 μA which is significantly lower than the peak magnitude P obtained in the graph in FIG. 1. This measurement result is considered due to the fact that magnesium sulfate contained in the buffer B is dissolved in the electrolyte solution, and its released Mg ion inhibits the reduction reaction. Specifically, the Mg ion present as a divalent ion in the electrolyte solution inhibits ionization of the ammonium molecule, to reduce the number of molecules of the free ammonium ion to be reduced by the reduction reaction, and as a result, the reduction current produced by the reduction reaction is decreased.

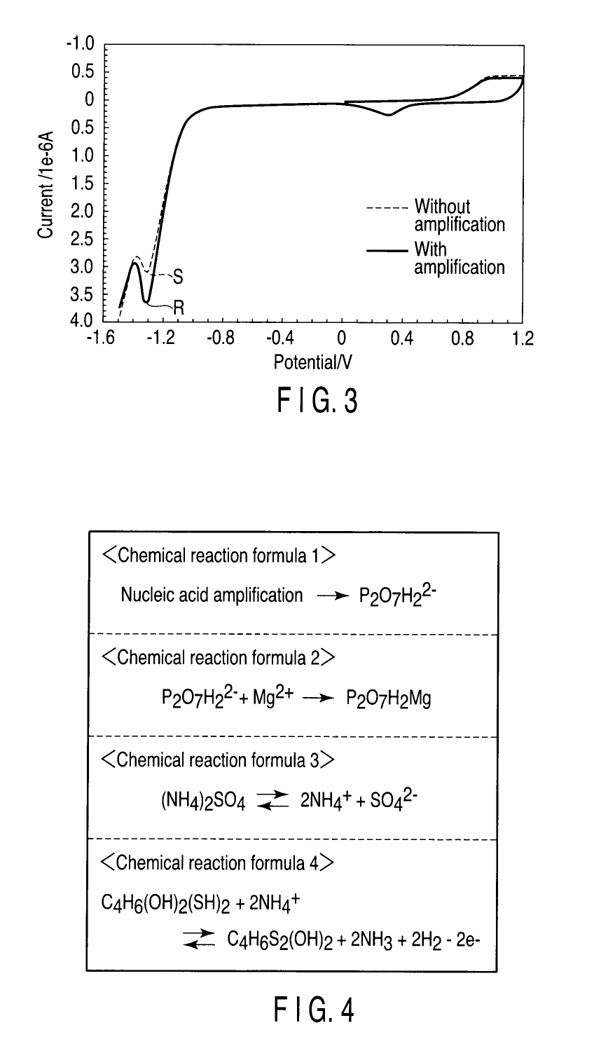

FIG. 3

FIG. 3 is a graph showing a measurement result of a reduction current produced by the reduction reaction in the first embodiment. In FIG. 3 similar to FIGS. 1 and 2, sweep potential is indicated on the abscissa and reduction current measurements on the ordinate. In measurement of reduction current, a sample nucleic acid is added to a buffer containing a reducing agent molecule, a redox molecule and magnesium sulfate in an electrolyte solution (Tris-HCl) in a container provided with a gold electrode, and a reduction current produced is measured with the gold electrode by cyclic voltammetry. The reducing agent molecule, the redox molecule and the potential sweeping method used are under the same conditions as in the graphs in FIGS. 1 and 2. Whether the sample nucleic acid had been amplified or not was separately confirmed based on the presence or absence of a band in electrophoresis. The graph in FIG. 3 shows that when the amplification reaction of the sample nucleic acid was conducted, a current of 3.6 μA is obtained as a peak current R (solid line) upon sweeping the potential to −1.3V or so. When the amplification reaction of the sample nucleic acid was not conducted, on the other hand, the peak current S obtained upon sweeping the potential to −1.3V or so is 3.1 μA (broken line). These measurement results mean that as the amplification reaction of the sample nucleic acid proceeds, the number of molecules of the free ammonium ion to be reduced by the reduction reaction is increased. This is due to the fact that the Mg ions in the solution are consumed by pyrophosphate ions formed with the progress of the amplification reaction of the sample nucleic acid.

FIG. 4

FIG. 4 is a table showing a series of chemical reaction formulae in the reduction reaction in the first embodiment.

In the amplification reaction of a nucleic acid, NTP substrates bind one after another to the corresponding complementary strand by the polymerase. At this time, a high-energy phosphate bond in the NTP substrate is utilized, and in conjunction with consumption of its energy, a pyrophosphate ion (P₂O₇H₂²⁻) is formed as a byproduct and released into the reaction solution (chemical reaction formula 1). The pyrophosphate ion released into the reaction solution is bound to an Mg ion to form magnesium pyrophosphate (P₂O₇H₂Mg), thus decreasing the Mg ion concentration of the reaction solution (chemical reaction formula 2). Meanwhile, the ammonium sulfate in the reaction solution is in a state of equilibrium with the free ammonium ion (chemical reaction formula 3). The above decrease in the Mg ion concentration of the reaction solution causes the above equilibrium state to be shifted to the side of the free ammonium ion (right side of the chemical reaction formula 3), to increase the free ammonium ion concentration of the solution. The ammonium ion is reduced with dithiothreitol (C₄H₆(OH)₂(SH)₂) as the reducing agent molecule in the reaction solution (chemical reaction formula 4), and a reduction current flowing upon this reduction is detected with the electrode. The reducing agent molecule dithiothreitol is present in excess in the reaction solution, so the magnitude of the flowing reduction current increases as the ammonium ion concentration of the reaction solution is increased.

That is, before the nucleic acid amplification occurs, the Mg ion present at high concentration in the reaction solution inhibits ionization of the ammonia molecule, and thus the obtained reduction current is low, but as the nucleic acid amplification reaction proceeds, the Mg ion concentration of the reaction solution is decreased so that the ionization of the ammonia molecule is promoted, and therefore, the obtained reduction current is increased. Accordingly, whether the nucleic acid amplification reaction has occurred or not can be determined on the basis of the magnitude of the obtained reduction current.

By sweeping the potential once in the direction of oxidation of an ammonium ion as the redox molecule to sufficiently oxidize the ammonium ion and then sweeping the potential in the direction of reduction of the ammonium ion, the equilibrium state of the reduction reaction can be shifted maximally to the direction of reduction of the ammonium ion, and as a result a larger reduction current can be obtained, as described above. At this time, the potential is swept preferably to −2 to 0V in the direction of reduction.

(3-2) Apparatus for Measuring Reduction Current

The electrochemical measurement in the first embodiment can be conducted using a potentiostat. The method of electrochemical measurement includes, for example, pulse techniques such as CV, LSV and DPV, as well as CC, CA, and impedance measurement. In these techniques, a measurement apparatus, a measurement container, and the arrangement of electrodes are not particularly limited as long as the electrochemical measurement is possible. Hereinafter, examples of the measurement apparatus used in the first embodiment are shown. Actually, all measurement apparatuses including various modifications and improvements thereof can be used in the method of the present invention.

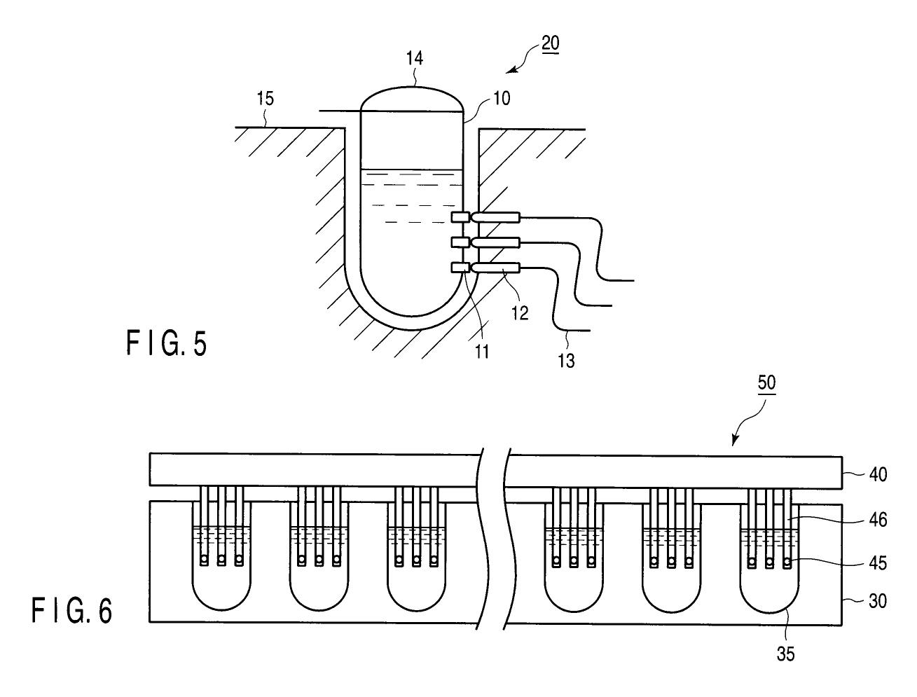

FIG. 5

FIG. 5 is a schematic diagram of a first measurement apparatus 20 used in the first embodiment. The first measurement apparatus 20 is characterized in that the amplification reaction of a sample nucleic acid and the measurement of a reduction current produced by reduction reaction are conducted in the buffer in the same container (amplification reaction tube 10) provided with an electrode. The amplification reaction and the measurement of a reduction current can be conducted in the buffer in the same container, thus simplifying the apparatus structure and realizing easy detection.

The amplification reaction tube 10 includes an electrode 11 embedded in the wall surface thereof, and can measure the reduction current of a solution in the tube. In FIG. 5, the electrode is indicated as the electrode 11 of three-electrode type. When the amplification reaction tube 10 is set in an amplification apparatus 15, the electrode 11 of three-electrode type is contacted with a terminal 12 built into the amplification apparatus 15, thus enabling measurement of the current in the solution in the tube 10. Each terminal 12 is connected via a lead wire 13 to the reduction current-measuring apparatus.

The electrode 11 is preferably of a three-electrode type, but may be of a two- or four-electrode type. Because a faint reduction current is measured in the method in accordance with the first embodiment, the material for the electrode is not particularly limited, and highly electroconductive arbitrary materials, for example gold, platinum, silver, copper, aluminum, nickel, iron and carbon can be used. Particularly, gold electrodes are highly reliable and can be supplied stably in a large amount, and are thus suitable for use in clinical tests and medical field. The electrode can be subjected thereon to molecular modification to improve sensitivity. The molecular modification includes, for example, those modifications with mercaptans such as mercaptoethanol, mercaptohexanol, mercaptoheptanol, mercaptoethylene glycol, mercaptooligoethylene glycol, mercaptopolyethylene glycol an alkane thiol having a C30 to C50 chain, lipids such and stearylamines, surfactants, albumins and nucleic acids.

In the measurement procedures, a nucleic acid amplification buffer is first injected into an amplification reaction container 10, and a sample nucleic acid is then added. A lid 14 of the amplification reaction container 10 containing the nucleic acid amplification buffer and the sample nucleic acid is closed and sealed, and the container 10 is then set in an amplification apparatus 20. At this time, the reduction current of the solution in the container 10 may be measured to determine a standard current of the sample nucleic acid before amplification. Subsequently, the solution in the container 10 is heated by the amplification apparatus 20. This heating operation is appropriately changed depending on the amplification method, reaction conditions, etc. After heating, the reduction current of the solution in the container 10 is measured.

FIG. 6

FIG. 6 is a schematic diagram of a second measurement apparatus 50 used in the first embodiment. The second measurement apparatus 50 is characterized in that a plurality of sample nucleic acids are simultaneously subjected to amplification reaction on a microtiter plate, and reduction currents of the plurality of sample nucleic acids are measured with a measurement substrate equipped with a plurality of measurement electrodes corresponding to wells of the microtiter plate. The second measurement apparatus 50 makes use of a microtiter plate-type amplification reaction container and its corresponding measurement substrate thereby enabling simultaneous detection of a plurality of samples and is thus extremely useful in clinical tests and medical field.

FIG. 6 is a schematic diagram of a cross section of one arbitrary lane of the microtiter plate 30. A sample nucleic acid and a nucleic acid amplification buffer are injected to each well 35 of the microtiter plate 30 to initiate an examination. After amplification reaction, the reduction current of the solution in each well 35 is measured. Measurement of the reduction current is conducted using a measurement substrate 40 equipped with measurement electrodes 45 corresponding respectively to the wells 35 of the microtiter plate 30. By mounting the measurement substrate 40 on the microtiter plate 30, the measurement electrode 45 fixed to the measurement substrate 40 is dipped in the solution in each well 35 of the microtiter plate 30. The measurement electrode 45 is preferably fixed in the vicinity of the top of a thin electrode support 46 so as to be certainly dipped in the solution in each well 35. In FIG. 6, the measurement electrode 45 is indicated as three-electrode type, but is not limited thereto as described above. The measurement substrate 40 is connected to the reduction current-measuring apparatus and can simultaneously measure reduction currents of the solutions in the wells 35, whereby a plurality of sample nucleic acids can be simultaneously determined for the presence or absence of their amplification.

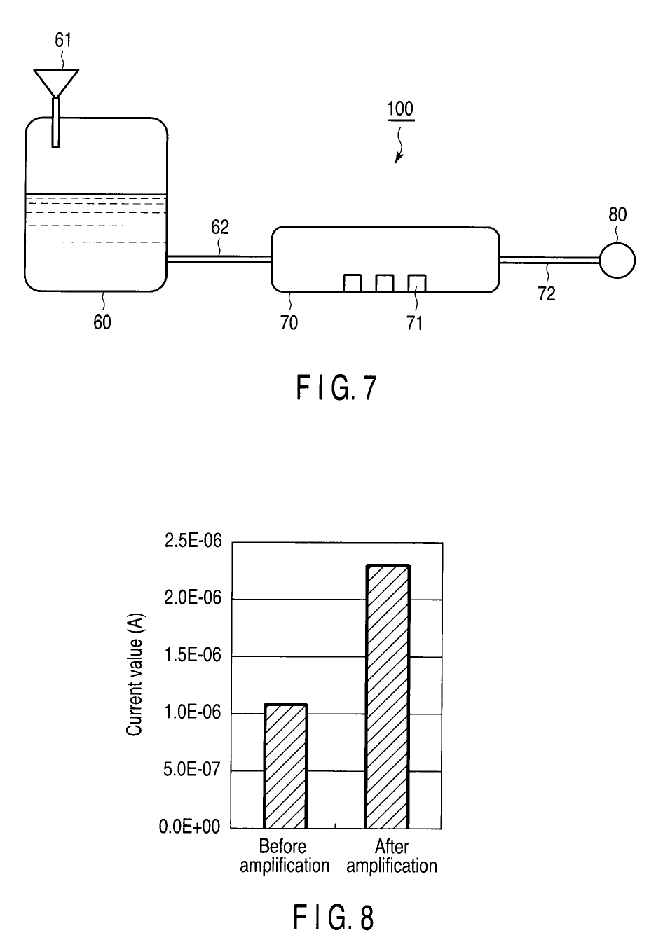

FIG. 7

FIG. 7 is a schematic diagram of a third measurement apparatus 100 used in the first embodiment. The third measurement apparatus 100 is characterized in that the amplification treatment of a sample nucleic acid is conducted in a first container for nucleic acid amplification (amplification chamber 60), and after the amplification treatment of the sample nucleic acid, the sample nucleic acid is transferred to a second container (detection chamber 70) provided with electrodes, and the measurement of a reduction current produced by the reduction reaction is conducted in the second container. Generally, the shape of a chamber suitable for amplification is different from the shape of a chamber suitable for detection. The amplification chamber 60 and the detection chamber 70 are formed separately so that the apparatus structure can have many variations to easily provide the optimum detection system for the amplification reaction and the detection reaction, respectively. Particularly, the detection chamber 70 can be a shallow flat-bottomed container to reliably and accurately detect the reduction current of the solution as a whole. Because the detection chamber requires electrodes to make itself relatively expensive, the amplification chamber only can be disposed after used once, while the detection chamber 70 can be repeatedly used, thus reducing the detection cost.

The amplification chamber 60 is provided with an injection opening 61, and a sample nucleic acid and a nucleic acid amplification buffer injected through the injection opening 61 are subjected to amplification treatment in the amplification chamber 60. The amplification chamber 60 is connected via a flow pass 62 to the detection chamber 70, so after the amplification treatment, the amplification reaction solution is sent through the flow pass 62 into the detection chamber 70. The detection chamber 70 is provided with a measurement electrode 71 by which the reduction current of the solution in the detection chamber 70 can be measured. In FIG. 7, the measurement electrode 71 is indicated as three-electrode type, but is not limited thereto as described above. The detection chamber 70 is connected via a flow pass 72 to a discharge opening 80. It follows that when amplification of the sample nucleic acid is confirmed as a result of measurement of the reduction current in the detection chamber 70, the amplified sample nucleic acid is recovered from the discharge opening 80 through the flow pass 72.

In a modified example of the third measurement apparatus 100, two or more amplification chambers 60 may be connected to the upstream part of the detection chamber 70. By connecting two or more amplification chambers 60, a plurality of sample nucleic acids can be successively detected in one detection chamber 70.

### (4) Determination of the Presence or Absence of Nucleic Acid Amplification Reaction

The magnitude of the reduction current accurately reflects the presence or absence of the nucleic acid amplification reaction. It follows that by previously determining a threshold magnitude of the reduction current flowing after the amplification reaction under the same reaction conditions, it can be determined that the amplification reaction has sufficiently occurred when a reduction current higher than the threshold magnitude is detected, while it can be determined that the amplification reaction has not occurred at all or has not occurred sufficiently when a reduction current higher than the threshold magnitude is not detected.

The reduction current before the amplification reaction may be obtained by measuring the reduction current before the amplification reaction. At this time, it can be determined that the amplification reaction has occurred sufficiently when the reduction current after the amplification reaction is significantly increased as compared with the reduction current before the amplification reaction, while it can be determined that the amplification reaction has not occurred at all or has not occurred sufficiently when the reduction current is hardly changed or a sufficiently high current is not obtained after the amplification reaction. Also in this case, accurate and rapid determination is possible by previously determining a threshold magnitude. When an approximate reduction current before the amplification reaction is known, the measurement of the reduction current before the amplification reaction can be omitted.

The method in the first embodiment is particularly effective for example when a nucleic acid detection process for detecting a sequence of an amplification nucleic acid product is to be conducted after nucleic acid amplification. That is, if the reduction current is measured after the amplification treatment of a sample nucleic acid, and from the measurement result, it is determined that the amplification of the sample nucleic acid has not sufficiently occurred, then the nucleic acid amplification product to be detected is estimated to be not present in a sufficient amount, and thus the nucleic acid detection process becomes unnecessary. Accordingly, when the presence or absence of a certain virus for example is detected, unnecessary labor hour and examination time can be significantly eliminated.

### EXAMPLES

### Example 1

### 1. Materials Used, Etc.

(1) Nucleic Acid Amplification Buffer

In the nucleic acid amplification buffer, a primer set for model gene A amplification shown below, an enzyme for LAMP amplification, and a buffer were used. As a template, sample nucleic acid V of unknown sequence was used.

Model Gene A: Mouse 2C39

Primer Set for Model Gene A Amplification

TCAAAACGATCCTGGAAAATAATGGACATTCATTCTGAGCTGTGC
GGAAAAACTAAATGAGAATGTCAAGCAGAAAAAACATTCTTGACTTC
TTCACGCTCACCTTGTGA
CTGTGGCAATAAAGCACC
AGCAGATGACATTGCATGGA

(2) Electrodes

A working electrode, a counter electrode and a reference electrode used as electrochemical measurement electrodes were gold electrodes.

### 2. Experimental Procedures

First, an amplification reaction solution was prepared. The primer set for model gene A amplification, the enzyme for LAMP amplification and the buffer were mixed, and the sample nucleic acid V was added as a template.

Then, electrochemical measurement was conducted before the initiation of amplification. Cyclic voltammetry was used as the measurement method. The potential was swept once from 0 to 1.2V in the direction of oxidation and then swept to −1.6V in the direction of reduction. The sweep speed was 0.3 V/s.

Thereafter, the amplification reaction was conducted at an amplification temperature of 63° C. for 60 minutes.

After the amplification, electrochemical measurement was conducted again. The measurement conditions were the same as before the amplification.

### 3. Experimental results

FIG. 8 is a graph showing peak magnitudes of reduction currents obtained before and after the amplification. The peak current before the amplification step was 1.08 μA, and the peak current after the amplification step was 2.31 μA. Both the peak currents are those currents from which the background current was subtracted (this hereinafter applies). After the amplification step, a current higher than a threshold magnitude of 1.20 μA was obtained, and it was thus found that the amplification of the sample nucleic acid V had significantly occurred, and that a sequence of the model gene A had been contained in the sample nucleic acid V. According to separately conducted analysis of the sample after the amplification, a band was confirmed in electrophoresis, and it was thus confirmed that the amplification had certainly occurred.

### Example 2

### 1. Materials Used, Etc.

(1) Nucleic Acid Amplification Buffer

In the nucleic acid amplification buffer, primer sets for model genes A and B amplification shown below, an enzyme for LAMP amplification, and a buffer were used. As templates, sample nucleic acids X and Y of unknown sequence were used.

Model Gene A: Mouse 2C39

Primer Set for Model Gene A Amplification

TCAAAACGATCCTGGAAAATAATGGACATTCATTCTGAGCTGTGC
GGAAAAACTAAATGAGAATGTCAAGGAGAAAAAACATTCTTGACTTC
TTCAGGCTCACCTTGTGA
CTGTGGCAATAAAGCACC
AGCAGATGACATTGCATGGA

Model Gene B: Mouse 481

Primer Set for Model Gene B Amplification

ATTTGGAACATACTGCTCTCTTCTGCTGCCATCTTCCTTTTGACA
AACTCAGACCTCCTTGAAAAGAACACAAAATCCTCGATAACTCGG
ATCTGGGAAGGATCAGCC
TGTCTGAAGATAGCTATTCACA

(2) Electrodes

A working electrode, a counter electrode and a reference electrode used as electrochemical measurement electrodes were gold electrodes.

### 2. Experimental Procedures

First, amplification reaction solutions were prepared. In amplification reaction solution 1, the primer set for model gene A amplification, the enzyme for LAMP amplification and the buffer were mixed, and the sample nucleic acid X was added as a template. In amplification reaction solution 2, the primer set for model gene A amplification, the enzyme for LAMP amplification and the buffer were mixed, and the sample nucleic acid Y was added as a template. In amplification reaction solution 3, the primer set for model gene B amplification, the enzyme for LAMP amplification and the buffer were mixed, and the sample nucleic acid X was added as a template. In amplification reaction solution 4, the primer set for model gene B amplification, the enzyme for LAMP amplification and the buffer were mixed, and the sample nucleic acid Y was added as a template.

Thereafter, the amplification reaction was conducted at an amplification temperature of 63° C. for 60 minutes.

After the amplification, electrochemical measurement was conducted in each of amplification reaction solutions 1 to 4. The measurement conditions were the same as in Example 1.

### 3. Experimental Results

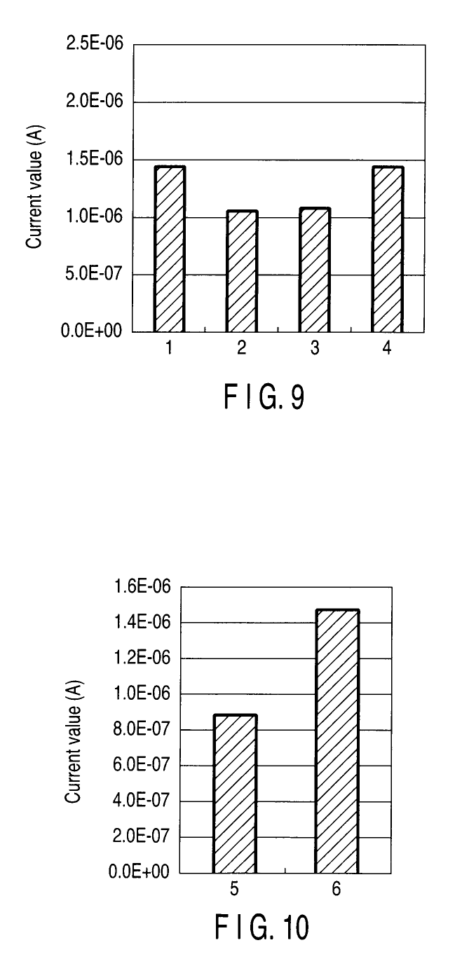

FIG. 9 is a graph showing peak magnitudes of reduction currents obtained in amplification reaction solutions 1 to 4. The peak currents in amplification reaction solutions 1 to 4 were 1.48, 1.02, 1.05 and 1.44 μA, respectively. In amplification reaction solutions 1 and 4, currents higher than the threshold magnitude of 1.20 μA were obtained, and it was thus found that sufficient amplification had occurred in amplification reaction solutions 1 and 4, and the sample nucleic acid X had a sequence of the model gene A and the sample nucleic acid Y had a sequence of the model gene B. When amplification reaction solutions 1 to 4 were observed in separately conducted electrophoresis, a band was confirmed in amplification reaction solutions 1 and 4, while a band was not confirmed in the reaction solutions 2 and 3, and it was thus confirmed that sufficient amplification had occurred in amplification reaction solutions 1 and 4 only.

### Example 3

### 1. Materials Used, Etc.

(1) Nucleic Acid Amplification Buffer

In the nucleic acid amplification buffer, primer sets for model genes C and D amplification shown below, an enzyme for PCR amplification, and a buffer were used. As a template, sample nucleic acid Z of unknown sequence was used.

Model Gene C: NAT481

Primer Set for Model Gene C Amplification

CTATTTTTGATCACATTGTAAGAAG
GCTCTCTCCTGATTTGGT

Model Gene D: NAT590

Primer Set for Model Gene D Amplification

ATACTTATTTACGCTTGAACCTC
GTTCCTTATTCTAAATAGTAAGGGAT

(2) Electrodes

A working electrode, a counter electrode and a reference electrode used as electrochemical measurement electrodes were gold electrodes.

### 2. Experimental Procedures

First, amplification reaction solutions were prepared. In amplification reaction solution 5, the primer set for model gene C amplification, the enzyme for PCR amplification and the buffer were mixed, and the sample nucleic acid Z was added as a template. In amplification reaction solution 6, the primer set for model gene D amplification, the enzyme for PCR amplification and the buffer were mixed, and the sample nucleic acid Z was added as a template.

Thereafter, the amplification reaction was conducted under the following temperature conditions:

(i) Step 1: 95° C., 5 min.

(ii) Step 2: 98° C., 10 s.

(iii) Step 3: 65° C., 30 s.

(iv) Step 4: steps 2 and 3 are repeated in 40 cycles.

(v) Step 5: 72° C., 1 min.

After the amplification, electrochemical measurement was conducted in amplification reaction solutions 5 and 6. The measurement conditions were the same as in Example 1.

### 3. Experimental Results

FIG. 10 is a graph showing peak magnitudes of reduction currents obtained in amplification reaction solutions 5 and 6. The currents obtained in amplification reaction solutions 5 and 6 were 0.89 and 1.43 μA, respectively. In amplification reaction solution 6, a current higher than the threshold magnitude of 1.20 μA was obtained, and it was thus found that sufficient amplification had occurred, and a sequence of the model gene D had been contained in the sample nucleic acid Z. In separately conducted electrophoresis, a band was not confirmed in amplification reaction solution 5, while a band was confirmed in amplification reaction solution 6, and it was thus confirmed that the amplification reaction had occurred in amplification reaction solution 6 only.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method of detecting a nucleic acid amplification reaction, comprising the steps of:
adding a sample nucleic acid to a nucleic acid amplification buffer containing a reducing agent molecule, a redox molecule and a magnesium ion, to conduct an amplification reaction;
measuring a reduction current produced by a reduction reaction of the reducing agent molecule with the redox molecule, under the conditions that when the amplification reaction of the sample nucleic acid has proceeded in the buffer, pyrophosphoric acid formed with the progress of amplification of the sample nucleic acid forms magnesium pyrophosphate with the magnesium ion, thereby decreasing a magnesium ion concentration of the buffer; and
determining, from a magnitude of the reduction current measured above, whether the sample nucleic acid has been amplified or not.

2. The method according to claim 1, wherein the step of measuring a reduction current comprises a step of sweeping a potential in a direction of oxidation of the redox molecule and then sweeping the potential in a direction of reduction, so as to induce the reduction reaction of the reducing agent molecule with the redox molecule, and a step of measuring a reduction current produced by the reduction reaction upon sweeping the potential in the direction of reduction.

3. The method according to claim 2, wherein the potential is swept to −2 to 0V in the direction of reduction.

4. The method according to claim 1, wherein the reducing agent molecule is dithiothreitol (DTT) or β-mercaptoethanol.

5. The method according to claim 1, wherein the redox molecule is a molecule to be reduced only in the presence of the reducing agent molecule.

6. The method according to claim 1, wherein the redox molecule is a molecule having an unshared electron pair.

7. The method according to claim 1, wherein the redox molecule is an ammonium ion.

8. The method according to claim 1, wherein the amplification reaction is selected from the group consisting of PCR, LAMP, ICAN and SMAP.

9. The method according to claim 1, wherein the step of measuring a reduction current comprises detection with a potentiostat.

10. The method according to claim 1, wherein the amplification reaction of the sample nucleic acid and the measurement of a reduction current produced by the reduction reaction are conducted in the buffer in the same container provided with electrodes.

11. The method according to claim 1, wherein a plurality of sample nucleic acids are simultaneously subjected to amplification reaction on a microtiter plate, and reduction currents of said plurality of sample nucleic acids are measured simultaneously with a measurement substrate equipped with a plurality of measurement electrodes corresponding to wells of the microtiter plate.

12. The method according to claim 1, wherein amplification treatment of the sample nucleic acid is conducted in a first container for nucleic acid amplification, after which the sample nucleic acid is transferred to a second container provided with electrodes, and the measurement of a reduction current produced by the reduction reaction is conducted in the second container.
